(19) United States
(12) Patent Application Publication
(10) Pub. No.: US 2010/0206751 A1
(43) Pub. Date: Aug. 19, 2010

(54) SUPPLY ORGANIZERS AND METHODS FOR HEALTH MANAGEMENT

(75) Inventor: Paul Wessel, Delano, MN (US)

Correspondence Address: Dugan & Dugan, PC, 245 Saw Mill River Road, Suite 309, Hawthorne, NY, 10532, US

(73) Assignee: Bayer HealthCare LLC, Tarrytown, NY (US)

(21) Appl. No.: 12/389,230

(22) Filed: Feb. 19, 2009

(51) Int. Cl. A61B 19/02; A45C 15/00

(52) U.S. Cl. 206/38; 206/370

(57) ABSTRACT

In some aspects, a health management supply organizer is provided. The supply organizer is adapted to receive health management supplies including an analyte meter and analyte sensors (e.g., glucose test strips) and a removable disposal container which is adapted to receive used analyte sensors therein. The removable disposal container is mounted on an end portion of the body and may include a viewing window. Numerous other aspects are provided.

### FIELD OF THE INVENTION

The present invention relates to supply organizers for health management, and methods of organizing such supplies.

### BACKGROUND OF THE INVENTION

The monitoring of analyte concentration levels in bio-fluids (e.g., blood, urine, etc.) may be an important part of health management (including health testing and/or control). For example, analyte sensors (sometimes referred to as “test strips”) may be used for the monitoring of a patient's blood glucose level as part of diabetes testing. In blood glucose testing, for example, the patient may use a portable lancing device which may be a spring-loaded, trigger-releasable device which receives a single-use, disposable lancet. When the lancet is released, a sharp portion of the lancet may prick the user's body part to produce a droplet of blood. That blood droplet may then be transferred to a test strip which interfaces with, and may be received within, a port in a blood glucose meter. The test strips may also be single use and disposable.

Depending on the reading obtained from the meter, a user may need to undertake control measures, such as by taking a glucose tablet or administering insulin. The user may also need to carry, and have available for use, other monitoring (testing) supplies, such as logbooks (diaries), analyte sensor vials, extra analyte sensors, extra lancets, control or normal solutions, and other monitoring supplies. Furthermore, the user may need to carry control supplies such as insulin, glucose tablets and/or insulin delivery devices (e.g., syringes, pens, pen needles), and/or other control supplies.

It would, therefore, be beneficial to provide a system for containing and organizing such health management supplies (monitoring and/or control supplies) to allow for compact storage of, and easy access to, such supplies.

### SUMMARY OF THE INVENTION

In a first aspect, the present invention provides a health management supply organizer, including a body adapted to receive health management supplies including an analyte meter and analyte sensors, the body having a first leaf and a second leaf and an end portion connecting the first leaf and the second leaf; and a removable disposal container mounted to the body along the end portion which is adapted to receive used analyte sensors.

In another aspect, the present invention provides a health management supply organizer, including a closeable body adapted to receive health monitoring supplies including an analyte meter and analyte sensors, the closeable body including a first leaf and a second leaf and an end portion connecting the first leaf and the second leaf, wherein the first leaf and the second leaf are foldable about the end portion; and a removable disposal container mounted to the body by being received in a pocket formed along the end portion, the removable disposal container including a tubular body and an end cap wherein one of the tubular body and the end cap include an opening adapted to receive used analyte sensors.

In a method aspect, the present invention provides a method of organizing health supplies, including providing a closeable body adapted to contain health management supplies including at least an analyte meter and analyte sensors; and providing a removable disposal container on the body wherein the disposal container is adapted to receive used analyte sensors.

In another method aspect, the present invention provides a method of organizing health management supplies, including providing a closeable body having a first leaf, a second leaf and an end portion connecting the first leaf and the second leaf, the closeable body adapted to contain health management supplies including at least an analyte meter and analyte sensors; and providing a removable disposal container on the end portion of the body wherein the disposal container is adapted to receive used analyte sensors.

Other features and aspects of the present invention will become more fully apparent from the following detailed description, the appended claims and the accompanying drawings.

### BRIEF DESCRIPTION OF THE DRAWINGS

FIG. 1 is a top plan view of an exemplary embodiment of a health management supply organizer provided according to the present invention, shown in an opened configuration.

FIG. 2 is a left end view of the supply organizer of FIG. 1, shown in a closed configuration.

FIG. 3 is a top plan view of the supply organizer of FIG. 1, shown in a closed configuration.

FIG. 4 is a side view of a removable disposal container of the supply organizer of FIG. 1.

FIG. 5 is a cross-sectional side view of the removable disposal container taken along line 5-5 of FIG. 4.

FIG. 6 is a left end view of another exemplary embodiment of a health management supply organizer provided according to the present invention, shown in a closed configuration.

FIG. 7 is a top plan view of the supply organizer of FIG. 6, shown in a closed configuration.

FIG. 8 is a top plan view of the supply organizer of FIG. 6, shown in an open configuration.

FIG. 9 is a side view of a removable disposal container of the supply organizer of FIG. 6.

FIG. 10 is a cross-sectional side view of the removable disposal container taken along line 10-10 of FIG. 9.

FIG. 11 is a top plan view of another embodiment of supply organizer, shown in an open configuration.

### DETAILED DESCRIPTION

In the process of blood monitoring and/or control (health management), various supplies may be employed by the user. These supplies may include an analyte meter, sensor vials, analyte sensors (some types may be referred to as “test strips”), a lancing device, disposable lancets, and/or monitoring (testing) supplies, and devices and/or supplies for control (administering), such as insulin, glucose tablets, insulin pens, syringes, swabs, needles, cannulas, tubing, insertion devices, insulin patches, patch transmitters, cannulas, infusion pump cartridges, adapters, and/or infusion sets for patch pumps, etc. The term “analyte meter,” as used herein, includes a blood analyte meter, an insulin pump, a controller for an insulin delivery system, an insulin pump/glucose meter combination, a patch pump, a continuous glucose meter, or other like metering devices. The organizing system 100 may be used to organize supplies for monitoring and/or control of a blood analyte, such as glucose, hemoglobin Alc, lipids such as LDL, HDL and triglycerides, lactate, keytone, and other analytes.

Furthermore, in the process of health management, a byproduct of such testing and/or control is debris, such as used analyte sensors, used lancets, used insertion devices, needles, etc. Accordingly, disposal of this debris is of constant concern, as the debris may include bodily fluids (e.g., blood), and, thus may need to be appropriately disposed of. Furthermore, organization of the supplies associated with such health management is desirable so that such supplies are readily available in a neat, organized, and compact condition.

Accordingly, in a first aspect of the present invention, a health management supply organizer is provided. The exemplary health management supply organizer (hereinafter otherwise referred to as a “supply organizer,” or simply an “organizer” may include a closeable body having a first leaf and a second leaf foldable about an end portion and which is adapted to receive management supplies (e.g., analyte testing and/or control supplies). The supply organizer includes a removable disposal container mounted to the end portion of the body which is adapted to receive used analyte sensor strips and other debris from the blood monitoring and/or management processes. The removable disposal container may include a tubular body and an end cap. The tubular body may be transparent or translucent allowing the contents to be viewed, such as through a viewing window. The supply organizer may be adapted to carry and organize supplies (e.g., an analyte meter, analyte sensors, vials for storage of analyte sensors, disposable lancets, a lancing device, and/or devices for administering, etc.).

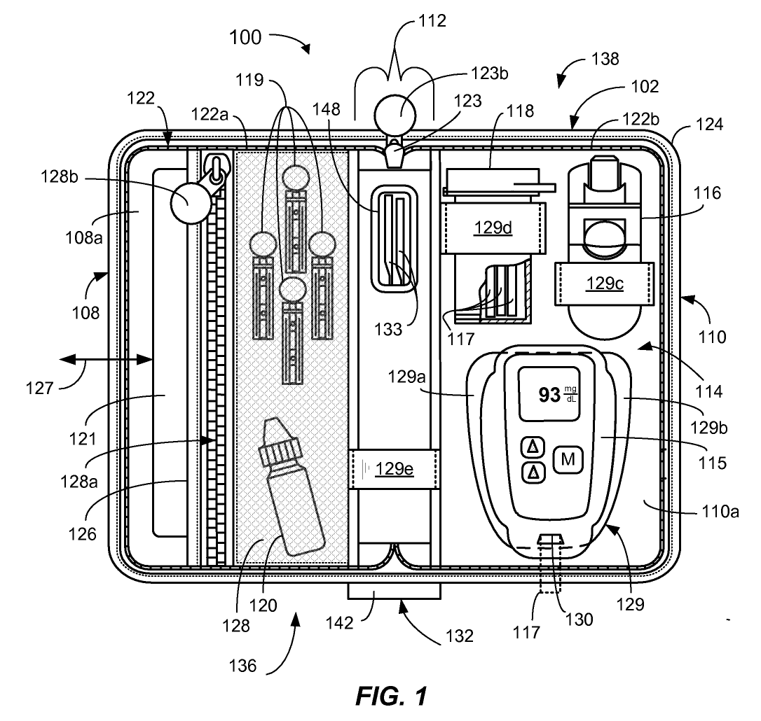

These and other embodiments of the health management supply organizer of the present invention are described below with reference to FIGS. 1-11.

FIGS. 1-5 illustrate a first exemplary embodiment of a health management supply organizer 100 according to the present invention. The supply organizer 100 may include a body 102 which may be closeable, and which may include a first leaf 108 and second leaf 110 which interconnect to, and may be foldable about, an end portion 112. The end portion 112 may be of any suitable construction and may be integral with the material forming the first leaf 108 and the second leaf 110, for example. The inside compartment 114 of the supply organizer 100 is adapted to receive testing and/or control supplies for analyte monitoring and/or control (i.e., receive the health management supplies).

In one embodiment, the health management supplies may be used for blood glucose management (e.g., monitoring and/or control). The monitoring supplies may include, but are not limited to, an analyte meter 115 (such as a blood analyte meter, insulin metering pump, etc.), a lancing device 116 such as an ASCENSIA® MICROLET® lancing device available from Bayer Healthcare LLC, disposable analyte sensors 117, a vial 118 for storage of the disposable sensors 117, disposable lancets 119, a dispenser bottle of control or normal solution 120, and a logbook 121, for example. The control supplies may further include delivery devices such as insulin pens or syringes, insulin, insulin supplies, swabs, tablets (e.g., glucose tabs), syringes, insertion devices, insulin patches, patch transmitters, cannulas, infusion pump cartridges, adapters, and/or infusion sets for patch pumps, etc. The body 102 may be manufactured from any suitable cloth or sheet material, such as nylon cloth material, plastic, leather or leatherette material, for example. Other materials may be used.

In some embodiments, the body 102 may be a hard material, such as molded plastic or metal. The inside compartment 114 may be accessed by, and closeable by a suitable closure member, such as a zipper assembly 122, for example. The zipper assembly 122 may include first and second zipper components 122a, 122b and a zipper hasp 123 adapted to engage the teeth of the respective zipper components 122a, 122b. The zipper components 122a, 122b may be sewed, or otherwise fastened to, respective panels 108a, 110a of the first leaf 108 and second leaf 110. The zipper hasp 123 may include a pull 123b coupled thereto which is adapted to be grasped by the user and aid in closure. The pull 123b may include an insignia of the manufacturer, for example. Zipper teeth on the zipper components 122a, 122b may be manufactured from any conventional materials such as metal or plastic. The panels 108a, 110a may be single-ply or multiple-ply (such as two- or three-ply, for example) and may include a different material on the outside and the inside. For example, the material on the inside may be a lighter weight material, for example. If a three-ply configuration, the center ply may be a reinforcement member or padding, such as a cardboard sheet, a plastic sheet, or a foam sheet. Optionally, the body 102 may include a finishing bead 124 sewn around at least a portion of the periphery, and in some embodiments, around the entire periphery. Other forms of closure mechanisms may be utilized (e.g., Velcro, snap, button, latch, magnets, etc.).

The inside compartment 114 may further include one or more pockets for receiving health management supplies. For example, one leaf, such as leaf 108 may include a first pocket 126 which is adapted to receive a logbook 121 which may be used for recording health information. The logbook may be removed from, and inserted in the pocket 126 along the direction of arrow 127. A second pocket 128 may be formed on, or as part of, a flap of the first pocket 126. For example, the second pocket 128 may include a mesh material which may be used to store disposable lancets 119, and/or control or normal solution 120, and/or other monitoring (testing) and/or control supplies described above. The use of a mesh material may allow the items inside the second pocket 128 to be viewed. Suitable mesh densities are about 100 mesh cells per square inch. Other cell densities may be used. The second pocket 128 may further include a closure member 128a, such as a conventional zipper assembly with pull 128b similar to the other zipper assembly described above. The second pocket 128 may be sewn, such as on its sides, or even on three sides into the panel 108a or otherwise secured to the first zipper component 122a. Optionally, the second pocket 128 may only be sewn to the end portion 112 or the panel 108a on the side closest to the end portion 112. The first pocket 126 may include a sewn bead or additional material wrapped around and sewn to its edge to provide a finished look to the edge.

The second leaf 110 may include a retainer 129 for receiving the analyte meter 115. The retainer 129 may be made of any suitable construction to receive and secure the analyte meter 115 in place within the inside compartment 114 of the organizer 100. For example, in one embodiment, the retainer 129 may be made from a flexible, molded plastic material (e.g., a thermoplastic elastomer) so that the analyte meter 115 may be readily snapped into and out of the retainer 129. Optionally, the retainer 129 may be a pocket configured and adapted to receive the analyte meter 115 and formed of any suitable sheet material, such as a cloth, elastic, mesh, leather, leatherette, or a transparent or translucent plastic sheet material. The retainer 129 may, in a simple embodiment, be formed as a strap of material, such as an elastic strap fastened at its ends to the panel 110a, for example. The retainer 129 may be sewn to or otherwise fastened, such as by adhesive or rivets, to the panel 110a. The retainer 129 may be oriented in any suitable fashion. In some embodiments, the analyte meter 115 may be oriented and the retainer 129 is configured such that a port 130 of the analyte meter 115, which is adapted to receive a disposable analyte sensor 117 (shown dotted), may be accessed by the disposable analyte sensor 117 without removing the analyte meter 115 from the retainer 129. Furthermore, the retainer 129 may be oriented and configured such that a display (e.g., a digital readout) of an analyte level of the tested bio-fluid is not obstructed and may be viewed by the user as the analyte meter 115 is mounted in the retainer 129. For example, a viewing window may be provided in the retainer 129 such as an opening or a clear panel which is aligned with the display of the meter. In the embodiment shown, the retainer may include a right flexible portion 129a and a left flexible portion 129b which are interconnected by a back portion, wherein the back portion is attached to the panel 110a, such as by rivets, sewing or adhesive.

Furthermore, the panel 110a may include one or more retainer loops or straps 129c, 129d adapted to receive and secure in place various other testing supplies. For example, a first retainer loop 129c may be adapted to receive and secure a lancing device 116 in place. The lancing device 116 may be any suitable lancing device known in the art, such as the ASCENSIA® MICROLET® lancing device available from Bayer Healthcare, LLC. Other types of lancing devices may be used. A second loop 129d may be included which may be adapted to receive and secure a vial 118 for storage of disposable analyte sensors 117. The vial 118 may be a conventional flip-top vial adapted to store analyte sensors, as known in the art. The straps 129c, 129d may be elastic or other compliant material and may be sewn with thread at their respective ends to the panel 110a, for example. Other loop configurations may be used. Pockets may also be used. The location of the pockets and retainers may be organized differently, such as by being switched, so that the pockets are on the second leaf 110 and the meter 115, etc. are mounted on the first leaf 108.

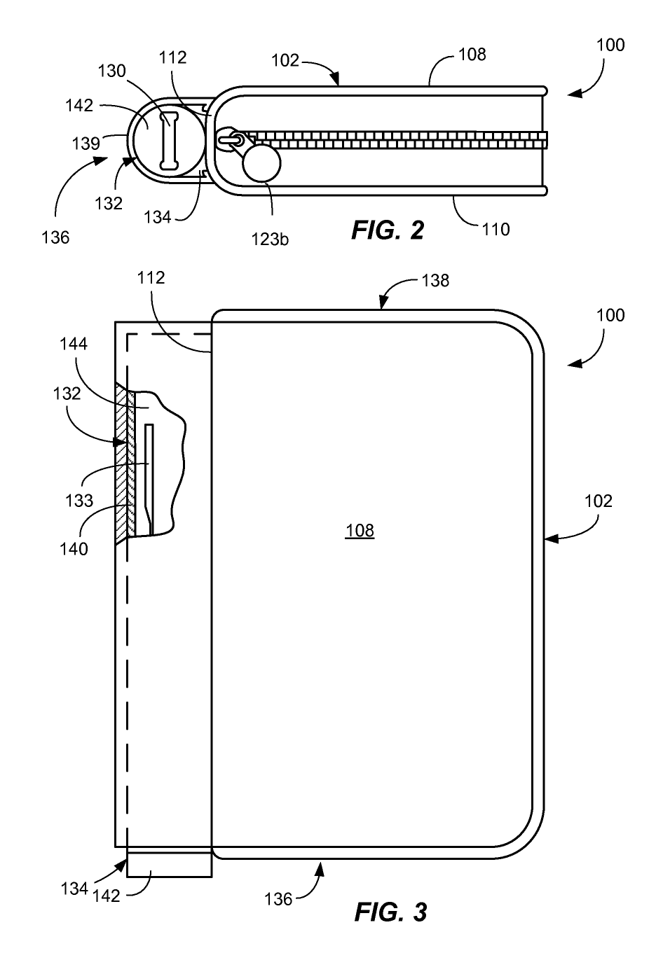

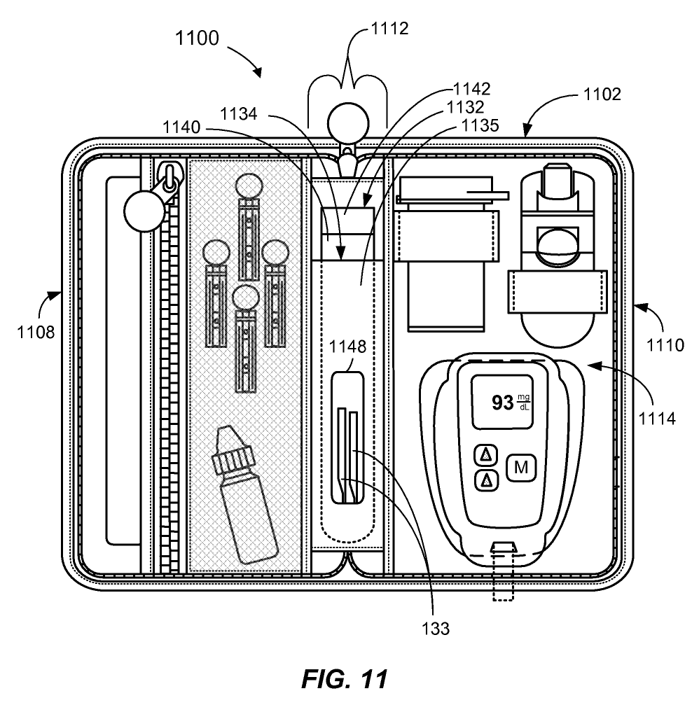

According to embodiments of the invention, the supply organizer 100 is further provided with a removable disposal container 132. The removable disposal container 132 may be mounted to the body 102 and is adapted to receive used analyte sensors 133. As best shown in FIG. 2, the removable disposal container 132 may be positioned along the end portion 112 of the body 102, and may be received in a pocket 134. The pocket 134 may be positioned along the end portion 112 and may extend from a first end 136 to a second end 138 of the body 102. The pocket 134 may be open on both ends or open on one end only. The pocket 134 may be formed in part of a separate piece of material 139 which may be secured to a portion of the body 102, such as the end portion 112 as shown in FIG. 2. The end portion 112 may form the other portion of the pocket 134. Optionally, the pocket 134 may be mounted inside of the body 102 along the end portion as shown in FIG. 11.

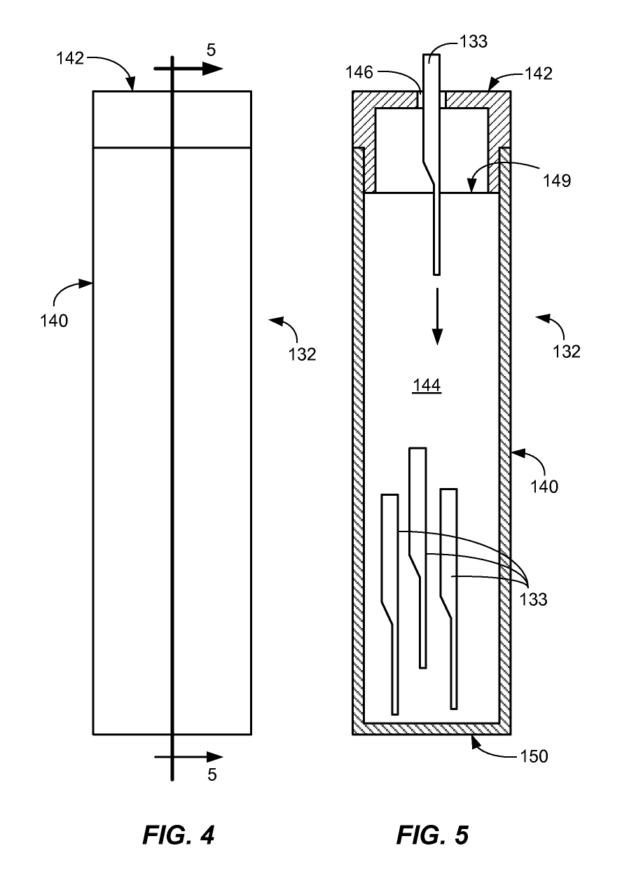

The removable disposal container 132, as best shown in FIGS. 4 and 5, may be closeable and may include a tubular body 140 and a cap 142. The cap 142 may be inserted in an open end of the tubular body 140 and may be removable such that the used analyte sensors 133 may be removed, from time to time, from the disposal cavity 144 of the removable disposal container 132. For example, the used analyte sensors 133, which may be considered a biohazard material, may be stored in the removable disposal container 132 for a time until the user can find a suitable waste receptacle in which to empty the contents of the disposal cavity 144. The cap 142 should be sufficiently tight so that it may not easily fall out of the tubular body 140. For example, the cap 142 may include a slight interference fit with the inside dimension of the tubular body 140. Optionally, the cap 142 may be threaded, hinged or otherwise fastened to the tubular body 140. The cap 142 may be plastic, rubber, thermoplastic elastomer, or cork, for example. Other suitable materials may be used.

In one or more embodiments, the removable disposal container 132 may include an opening 146, such as in a wall or an end face thereof. The opening 146 may be adapted to receive used analyte sensors 133 therein. In some embodiments, the opening 146 may be formed in the cap 142, for example. The opening 146 may be of any suitable shape and size, but in some embodiments may be made dimensionally just large enough to accommodate the dimensions of the used analyte sensors 133 received therein. The opening 146 should be not so large that the used analyte sensors 133 may easily fall out. For example, the opening 146 may have a cross-sectional area less than a cross-sectional area of the tubular body 140 when measured in transverse cross section. A flap of compliant material (not shown) may be formed or molded around or as part of the opening 146 to further prevent the used analyte sensors 133 from inadvertently falling out of the disposal cavity 144.

The tubular body 140 may have any suitable cross-sectional shape such as round, oval, square, rectangular, triangular, octagonal, hexagonal, etc. Further, the tubular body 140 may be made out of any suitable material. In some embodiments, at least a portion of the tubular body 140, and preferably all of the tubular body 140, may be constructed of a transparent or translucent material, for example. Materials such as transparent plastic (e.g., polyethylene terephthalate (PET), polypropylene (PP), or polystyrene (PS)), translucent plastic (e.g., High-density polyethylene (HDPE) or low-density polyethylene (LDPE)), tempered glass, or other like materials may be used. Opaque materials may also be used, such as plastic (e.g., polyvinyl chloride (PVC)), or metal (e.g., aluminum).

In the case where a transparent or translucent material is used, an amount of used analyte sensors 133 contained in the removable disposal container 132 may be viewed by the user by sliding the tubular body 140 out of the pocket 134 (FIG. 2). Optionally, the used analyte sensors 133 contained in the removable disposal container 132 may be viewed through a viewing window 148 formed in the body 102 as best shown in FIG. 1. For example, the viewing window 148 may be formed in the end portion 112 of the body 102 (FIG. 1). In other embodiments, an end portion of the tubular body 140 may extend through an opening between the pocket 134 and the inside compartment 114 such that at least a portion of the tubular body 140 is exposed inside of the organizer and is viewable by the user.

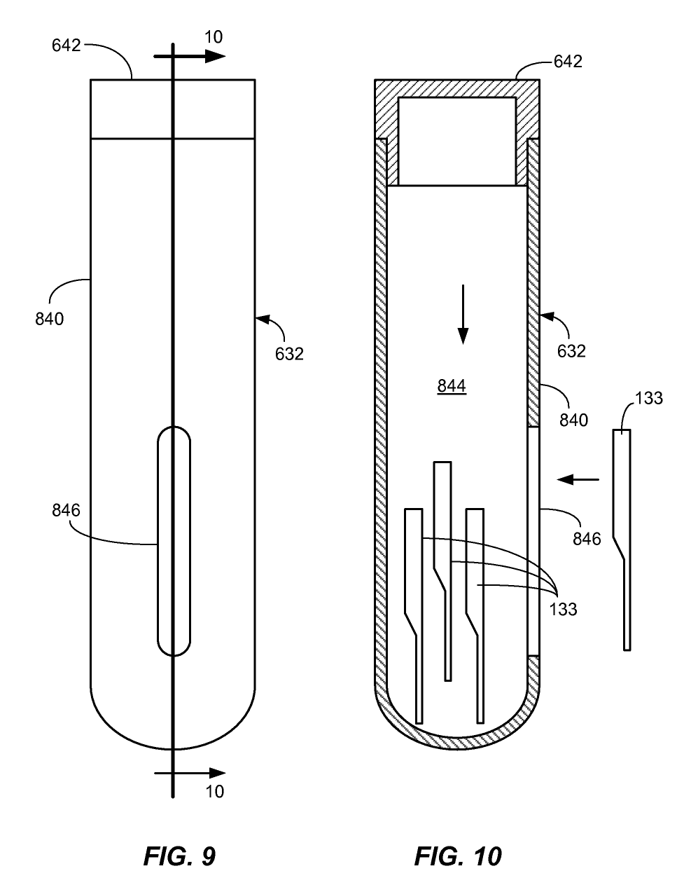

As best shown in FIGS. 4 and 5, the tubular body 140 may include an open end 149 into which the cap 142 is inserted and a closed end 150 which is positioned opposite the open end 149. The closed end 150 may include a 90-degree termination, such as shown in FIGS. 4 and 5, or may optionally include a rounded termination, much like a test tube, as shown in FIGS. 9 and 10. A length of the removable disposal container 132 may extend along the end portion 112, and a length of the removable disposal container 132 may be long in relation to its width, i.e., the removable disposal container 132 may be slender. For example, the removable disposal container 132 may have a test-tube shape with a length to width ratio of greater than 4, greater than 5, or greater than 6 or more. This may allow the removable disposal container 132 to be conveniently mounted in the body 102 in a way which minimally impacts the overall size (compactness) of the organizer 100.

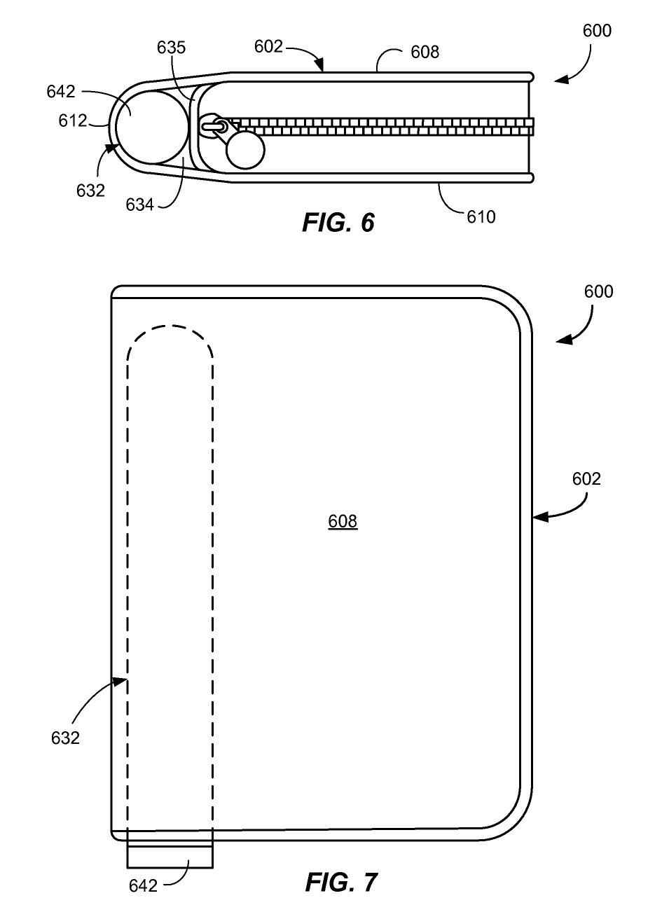

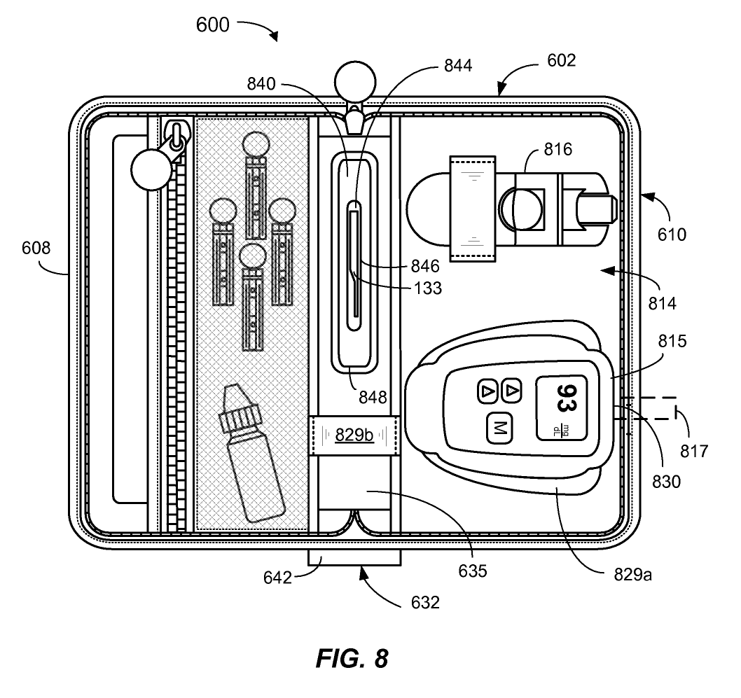

Another embodiment of the supply organizer 600 is shown in FIGS. 6-10. In this embodiment, the body 602 includes a single piece of material forming at least the outer portion of the first leaf 608, the second leaf 610 and the end portion 612, as best shown in FIG. 6. The removable disposal container 632 may be received in a pocket 634 formed along the end portion 612 and formed in part by the continuous piece of material. An inner flap of material 635, which may also extend along, and be secured to, the inside of the first leaf 608 and/or second leaf 610, may form the remainder of the pocket 634. Otherwise, this embodiment is the same as the previously-described embodiment except that the organizer 600 is adapted for use with a multi-sensor analyte meter 815 shown in FIG. 8, which may be an ASCENSIA® BREEZE® 2 Blood Glucose Meter available from Bayer Healthcare, for example. Other types of multi-sensor analyte meters may be used. A multi-sensor analyte meter includes a cartridge containing multiple analyte sensors wherein the cartridge is loaded into the analyte meter and the analyte sensors are adapted to be dispensed one at a time, as needed.

Accordingly, because the analyte sensors are housed within the meter 815, the vial 118 and the retainer loop 129d included in the previous embodiment of FIG. 1 may be removed. Additionally, the multi-sensor analyte meter 815 and a retainer 829 thereof may be oriented such that a port 830 of the analyte meter 815 may be disposed along a front of the second leaf 610 of the organizer 600, as depicted in FIG. 8. Other orientations may be used as well. In this orientation, an analyte sensor 817 (shown dotted) may be dispensed from the analyte meter 815 and may be accessed without needing to remove the analyte meter 815 from the organizer 600. Similarly, a lancing device 816 may be oriented such that it may be operated without removing the lancing device 816 from the organizer 600.

In this embodiment, the cap 642 does not include an opening. To provide improved access, an opening 846 into a disposal cavity 844 may be formed into a wall of the removable disposal container 632, as is best shown in FIGS. 8-10. The opening 846 may be of any suitable size or shape, but should be small enough so that the used analyte sensors 133 cannot easily fall back out of the removable disposal container 632. For example, the opening 846 may be an elongated slot having rounded corners which may extend along a length of a tubular body 840, as shown in FIG. 9. The opening 846 may be accessible from an inside compartment 814 (through a cutout viewing window 848 formed through a side of the pocket 634) of the organizer 600 as shown in FIG. 8. In particular, the opening 846 may be formed in the tubular body 840 of the container 632 and may be positioned such that it is aligned within the viewing window 848. In this manner, the used analyte sensors 133 may be inserted into the disposal cavity 844 through the opening 846. Furthermore, the contents of the removable disposal container 632 may be viewed by a user from the inside of the organizer 600 so that the user may make an easy determination of when to empty the removable disposal container 632. The removable disposal container 632 may be constructed as previously described.

In the depicted embodiments of FIGS. 1 and 8, the end portion 112 (FIG. 1) or the inner flap 635 (FIG. 8) of the organizers 100, 600 may include one or more members (e.g., 129e, 829b) forming pockets or elastic straps adapted to receive and secure an insulin delivery device (e.g., an insulin pen or syringe—not shown) onto the organizer 100, 600. To the extent that the organizer 100, 600 includes an insulin pen or syringe, the removable disposal container 132, 632 may be adapted to receive used sharps from these administering devices, as well. Furthermore, the removable disposal containers 132, 632 described herein may also be adapted to receive used disposable lancets 133 or other debris, such as used needles and/or used insertion devices.

Another embodiment of the supply organizer 1100 is depicted in FIG. 11. In this embodiment, the body 1102 is similar to the embodiments of FIGS. 1-5, in that a first leaf 1108 and a second leaf 1110 foldable about an end portion 1112 are included. However, in this embodiment, the removable disposal container 1132 may be received in a pocket 1134 formed on an inside of the end portion 1112, such that the disposal container is entirely housed within the inside compartment 1114 of the body 1102 and is hidden from view when the organizer 1100 is provided in a closed configuration. The pocket 1134 is formed by a cover piece of material 1135 which may also extend along, and be fastened (e.g., by stitching) to or along, the inside edges of the first leaf 1108 and second leaf 1110, for example. The pocket 1134 may be open at both ends or stitched at one, for example, as depicted in FIG. 11. Otherwise, this embodiment is the same as the previously-described embodiment shown in FIG. 1. Other types of analyte meters may be used with this embodiment by configuring the organizer as shown in FIG. 6, for example. As in the previous embodiments, a viewing window 1148 may be provided. For example, a portion of the cover piece of material 1135 may be cut out forming the viewing window 1148. Further, as in some previously-described embodiments, at least a portion of the tubular body 1140 may be transparent or translucent. As such, the used analyte sensors 133 received in the tubular body 1140 of the removable disposal container 1132 may be readily viewed. The viewing window 1148 allows an accumulation of the used analyte sensors 133 to be viewed so as to determine when to dispose of them. As in the previous embodiments, a removable cap 1142 may be employed, such that the used analyte sensors 133 may be removed and disposed of by the user from time to time. The cap 1142 may include an opening (not shown) such as shown in FIG. 2, for example. Optionally, a slot opening may be included in place of the cap opening, such as shown in FIG. 8.

The organizers 100, 600, 1100 described herein may include a handle (not shown), such as a tab, strap, carabineer, belt loop, fastener, or the like for being grasped by the user, or for securing the organizer 100, 600, 1100 to the user.

In accordance with another aspect of the invention, a method of organizing health supplies is provided. According to the method, an organizer with a closeable body having a first leaf and a second leaf foldable about an end portion may be provided. The organizer, which may be adapted to contain health management supplies, may include a removable disposal container provided on the body along the end portion, wherein the removable disposal container is adapted to receive one or more used analyte sensors. In one or more embodiments, the method may include inserting the removable disposal container into a pocket formed on the body. A cap may be inserted on a tubular body of the removable disposal container to form a disposal cavity. The method may further include a user inserting one or more used analyte sensors into the removable disposal container. Furthermore, the method may include dismounting the removable disposal container from the body, emptying the used analyte sensors from the removable disposal container, and thereafter remounting the removable disposal container on the body.

According to one or more embodiments, the supply organizer may include a body and one or more health management supplies which may be inserted into the body. For example, an analyte meter may be inserted into a retainer formed in an inside compartment of the body. Similarly, a lancing device may be inserted into a retainer member formed on the inside compartment of the body. Other health management supplies, as are mentioned above, may be inserted into the body.

The foregoing description discloses only exemplary embodiments of the invention. Modifications of the above-disclosed supply organizer which fall within the scope of the invention will be readily apparent to those of ordinary skill in the art. Accordingly, while the present invention has been disclosed in connection with exemplary embodiments thereof, it should be understood that other embodiments may fall within the spirit and scope of the invention, as defined by the following claims.

The invention claimed is:

1. A health management supply organizer, comprising:
a body adapted to receive health management supplies including an analyte meter and analyte sensors, the body having a first leaf and a second leaf and an end portion connecting the first leaf and the second leaf; and
a removable disposal container mounted to the body along the end portion which is adapted to receive used analyte sensors.

2. The health management supply organizer of claim 1 wherein the body is closeable and includes an inside compartment adapted to receive the analyte meter and analyte sensors.

3. The health management supply organizer of claim 1 wherein the body further comprises a viewing window adapted to allow viewing of contents inside the removable disposal container.

4. The health management supply organizer of claim 1 wherein a portion of the removable disposal container is transparent or translucent and adapted to allow at least a portion of disposal cavity of the container to be viewed.

5. The health management supply organizer of claim 1 wherein the removable disposal container is slidably received in a pocket.

6. The health management supply organizer of claim 5 wherein the removable disposal container is entirely contained in the organizer and is hidden from view when the organizer is in a closed configuration.

7. The health management supply organizer of claim 5 wherein the pocket is formed of a piece of material fastened along inside edges of the first leaf and second leaf.

8. The health management supply organizer of claim 5 wherein the pocket includes a viewing window.

9. The health management supply organizer of claim 1 wherein the removable disposal container further comprises a tubular body and a removable cap.

10. The health management supply organizer of claim 1 wherein one of the removable cap and the tubular body includes an opening adapted to receive the used analyte sensors.

11. The health management supply organizer of claim 10 wherein the tubular body includes a slot formed in a wall of the tubular body and the slot is adapted to receive the used analyte sensors.

12. The health management supply organizer of claim 1 wherein the removable disposal container further comprises a tubular body and a cap inserted in an end thereof, wherein the cap includes an opening adapted to receive the used analyte sensors.

13. The health management supply organizer of claim 12 wherein the body further comprises a viewing window adapted to allow viewing of contents inside the tubular body.

14. A health management supply organizer, comprising:
a closeable body adapted to receive health monitoring supplies including an analyte meter and analyte sensors, the closeable body including a first leaf and a second leaf and an end portion connecting the first leaf and the second leaf, wherein the first leaf and the second leaf are foldable about the end portion; and
a removable disposal container mounted to the body by being received in a pocket formed along the end portion, the removable disposal container including a tubular body and an end cap wherein one of the tubular body and the end cap includes an opening adapted to receive used analyte sensors.

15. A method of organizing health management supplies, comprising:
providing a closeable body having a first leaf, a second leaf and an end portion connecting the first leaf and the second leaf, the closeable body adapted to contain health management supplies including at least an analyte meter and analyte sensors; and
providing a removable disposal container on the end portion of the body wherein the removable disposal container is adapted to receive used analyte sensors.

16. The method of organizing health management supplies of claim 15, comprising inserting a used analyte sensor in the removable disposal container.

17. The method of organizing health management supplies of claim 15, comprising viewing an amount of used analyte sensors in the removable disposal container through a viewing window in the closeable body.

18. The method of organizing health management supplies of claim 15, comprising dismounting the removable disposal container from the closeable body, emptying the removable disposal container, and remounting the removable disposal container on the closeable body.

19. The method of organizing health management supplies of claim 15, comprising inserting the removable disposal container into a pocket formed in an inside compartment of the closeable body.

20. The method of organizing health management supplies of claim 15, comprising inserting a removable cap on the removable disposal container.
